(19) United States
(12) Patent Application Publication
(10) Pub. No.: US 2024/0108655 A1
(43) Pub. Date: Apr. 4, 2024

(54) METHODS OF PRODUCING MIXED CHIMERISM AFTER A SOLID ORGAN TRANSPLANT

(71) Applicant: MEDEOR THERAPEUTICS, INC., South San Francisco, CA (US)

(72) Inventors: Giovanni Ferrara, South San Francisco, CA (US); Chih Ping Liu, South San Francisco, CA (US)

(21) Appl. No.: 18/367,728

(22) Filed: Sep. 13, 2023

Related U.S. Application Data
(60) Provisional application No. 63/406,082, filed on Sep. 13, 2022.

(51) Int. Cl. A61K 35/17; A61K 35/28; A61N 5/10; C07K 16/24

(52) U.S. Cl. CPC A61K 35/17; A61K 35/28; A61N 5/10; C07K 16/24; A61K 2039/505

(57) ABSTRACT

The present invention provides methods used to condition recipients of organ transplants to achieve tolerance using a novel non-myeloablative conditioning regime featuring total lymphoid irradiation (TLI) and total body irradiation (TBI), each provided as a fractionated series of doses. Advantageously, this regime not only provides better conditioning than existing non-myeloablative regimes, but also does not require extending the typical duration of the conditioning course.

### FIELD OF THE INVENTION

The invention generally relates to methods for immunosuppressive conditioning of a transplant recipient that receives donor-derived blood cell progenitors.

### BACKGROUND

Nearly 35,000 organ transplants are performed in the United States each year. The primary complication of organ transplantation is rejection of the organ by the recipient's immune system. Advances in surgical technique and improved drugs to prevent infection and rejection have allowed transplantation of solid organs and/or composite tissues to become an effective treatment for many diseases. Transplanted organs include heart, intestine, liver, lung, pancreas and kidney.

A major barrier to organ transplantation arises from recipients' own immune systems, which often respond to a transplanted organ as “non-self” and reject it. This issue is more prevalent when the donor and recipient are non-HLA matched. Thus, successful transplantations often are followed by administration of medications to suppress a recipient's immune system. Immunosuppressive therapy, however, carries its own set of risks, including increased risk of infection, cancer, hypertension, metabolic disease and kidney and liver damage. In addition, immunosuppression does not guarantee that the recipient will tolerate the graft. Recipients usually receive a mixture of three maintenance immunosuppressive drugs, including a calcineurin inhibitor such as cyclosporine A, tacrolimus or sirolimus; prednisone; and an inhibitor of nucleic acid synthesis such as mycophenolate mofetil. The latter drugs have side effects that include hypertension, nephrotoxicity, susceptibility to infection, susceptibility to cancer and heart disease that contribute to long term patient disability, graft loss and even death. In spite of modern immunosuppressive drugs, in some centers acute rejection can occur in 10-25% of people after transplant.

It is therefore of great clinical interest to develop therapeutic regimens that achieve tolerance and minimization or complete withdrawal of immunosuppressive drugs in adult transplant patients. This approach has been explored in humans for HLA-matched transplantation, where the organ recipient becomes tolerant through co-transplantation of immune system cells (allogeneic hematopoietic cell transplantation, or HCT), which establish a state of chimerism. A danger exists, however, in the ability of the donor immune cells to generate a graft-versus-host disease. The development of persistent mixed chimerism and tolerance after bone marrow transplantation combined with organ transplantation in adult rodents and large laboratory animals has been achieved without graft versus host disease using non-myeloablative conditioning regimens.

Preclinical studies have shown that conditioning with total lymphoid irradiation (TLI) and anti-thymocyte globulin (ATG) is advantageous for inducing tolerance after combined organ and bone marrow transplantation because the conditioning regimen prevents GVHD as compared to total body irradiation (TBI). For a review, see Strober et al. (2011) Seminars in Immunology 23:273-281, which is incorporated by reference herein.

Nevertheless, given the deleterious effects of immunosuppressive medications, transplantation regiments with a reduced dependence on immunosuppressive medications would help alleviate many prevalent issues associated with transplants.

### SUMMARY

The present invention provides methods used to condition recipients of organ transplants to achieve tolerance using a novel non-myeloablative conditioning regime featuring total lymphoid irradiation (TLI) and total body irradiation (TBI), each provided as a fractionated series of doses. Advantageously, this regime not only provides better conditioning than existing non-myeloablative regimes, but also does not require extending the typical duration of the conditioning course.

As a means to prevent graft rejection, transplantation of organs may be accompanied by transfer of donor-derived blood cell progenitors, in particular, in cellular compositions comprising CD34+ cells derived from a donor. Providing donor blood cells allows reconstitution of the recipient's immune system to include cells from the donor of the organ that have been educated to recognize the organ as non-foreign tissue. Concurrently, GVHD is also avoided since the residual immune system of the recipient re-educates the immune system of the donor to recognize host tissue as well.

The present invention provides methods used to condition recipients of organ and tissue transplants who receive these donor-derived blood cell progenitors to achieve graft tolerance, and eventually achieve persistent mixed donor cell chimerism. Methods of the invention incorporate a novel non-myeloablative conditioning regime featuring total lymphoid irradiation (TLI) and total body irradiation (TBI), each provided as a fractionated series of doses. Advantageously, this regime not only provides better conditioning than existing non-myeloablative regimes, but also does not require extending the typical duration of the conditioning course.

The present invention is based on the insight that an immunosuppressive regime including a plurality of fractionated doses of both TLI and TBI suppresses a recipient's immune system to effectively facilitate engraftment, while also avoiding complete eradication of the recipient's immune system, which would prevent establishment of mixed chimerism.

In general, the methods of the invention include transplanting an organ or tissue from a donor into a recipient, performing a non-myeloablative conditioning regime of the invention, and infusing the recipient with a hematopoietic cell composition. The non-myeloablative conditioning regimes of the invention combine a plurality of TLI and TBI doses, i.e., at least two of each TLI and TBI. In particular, the total irradiation provided by the multiple TBI doses is far below the amount of irradiation generally used in TBI conditioning regimes—on the order of about between 30 and 150 cGy.

Accordingly, the present invention includes methods used in conjunction with organ transplantation. An exemplary method includes method for transplantation, the method comprising: conditioning the recipient of an organ transplant with a plurality of total lymphoid irradiation (TLI) doses, and at least two doses of total body irradiation (TBI), each dose of TBI between about 15 and about 75 cGy; infusing the recipient with a donor-derived hematopoietic stem cell product; wherein the recipient achieves stable, high level mixed-chimerism with the donor hematopoietic cells.

In preferred aspects, the method includes providing two doses of TBI to the recipient. Alternatively, three or more doses of TBI are provided to the recipient. In certain aspects, the plurality of TLI doses consists of between 1 and 11 doses. In preferred aspects, the plurality of TLI doses consists of 8 or 9 doses. Preferably, the plurality of TLI doses comprise a total dose of between about 1100 cGy and about 1300 cGy. Accordingly, each individual TLI dose comprises a dose of between about 110 and about 165 cGy. In certain aspects, the plurality of TLI doses comprise a total dose of about 1200 cGy. Thus, an individual TLI dose comprises a dose of about 110 cGy to about 150 cGy, and preferably from about 120 cGy to about 150 cGy.

In certain aspects, the methods of the invention provide 8 or 9 doses of TLI and 2 doses of TBI.

In certain aspects, the donor of the transplanted organ and the recipient are MHC-mismatched.

Methods of the invention may further include administering one or more doses of anti-thymocyte globulin (ATG) to the recipient. In certain methods, a final dose of irradiation is one or more of the TBI doses. In certain methods, the recipient receives a solid organ transplantation.

In certain methods, the donor of the organ transplant is the same donor that provided cell used to create the donor-derived hematopoietic cellular product. In preferred methods, the hematopoietic cellular product comprises CD34⁺ cells. In certain aspects, the hematopoietic cellular product further comprises CD3⁺ cells. In certain methods, the hematopoietic cellular product may comprise at least 1×10⁵ CD34⁺ cells/kg recipient weight and at least 1×10⁵ CD3⁺ cells/kg recipient weight. In certain methods, the hematopoietic cellular product may comprise at least 1×10⁶ CD34⁺ cells/kg recipient weight and at least 1×10⁶ CD3⁺ cells/kg recipient weight. In certain methods, the hematopoietic cellular product may comprise at least 4×10⁶ CD34⁺ cells/kg recipient weight and at least 1×10⁸ CD3⁺ cells/kg recipient weight.

In some methods, the hematopoietic cellular product comprises CD8⁺ memory T cells. The hematopoietic cellular product may include at least 1×10⁶ CD8⁺ memory T cells/kg recipient weight.

In some methods, the donor is living. Alternatively, methods of the invention may be used when the donor is deceased.

In preferred methods, prior the infusion of the hematopoietic stem cell product, the recipient receives a solid tissue or organ transplant. In some methods, the recipient has an autoimmune disease. In certain methods, the recipient is afflicted with a cancer.

Compositions comprising CD3+ and CD34+ cells that may be used in methods of the invention are described in U.S. Pat. Nos. 8,506,954; 9,114,157; 9,192,627; 9,290,813; 9,504,715; 9,504,717; 9,545,444; 9,561,253; 9,833,477; 9,974,807; 10,076,542; 10,080,769; 10,159,694; 10,166,256; 10,183,043; 10,256,648; 10,286,049; 10,549,082; 10,603,340; U.S. Patent Application Publication Nos. US 2016-0376653; US 2018-024337; US 2019-0083530; US 2019-0083537; US 2019-0091262; US 2019-0192561; US 2019-0192562; US 2019-0201514; US 2019-0275085; US 2019-0298762; US 2019-0307803; US 2019-0307804; US 2019-0336528; US 2019-0358257; US 2019-0307803; US 2020-0086004; US 2020-0087627; PCT International Application Publication Nos. WO/2012/096974; WO/2014/035695; WO/2014/133729; WO/2014/172532; WO/2019/178106; WO/2019/195657; WO/2019/195659; WO/2020/061180; PCT International Application No. PCT/US2019/051486; U.S. patent application Ser. Nos. 16/109,373; 16/573,387; 16/573,395; 16/573,408; 16/573,420; 16/716,102; and U.S. Provisional Application No. 62/858,142, the contents of each of which are herein incorporated by reference.

Compositions comprising CD3+ according to aspects of the present invention may also comprise hematopoietic facilitatory or human facilitating cells (hFCs). Advantageously the hFCs may be selected and provided to improve engraftment of the CD34+ cells. The compositions may comprise CD3+ cells and CD34+ cells, or CD3+ cells, CD34+ cells, and hFCs. CD3+ cells and CD34+ cells may be provided together in a single composition and the hFCs provided in a separate composition, or the CD3+ cells, CD34+ cells, and hFCs, may be provided together. hFCs are generally characterized as CD8+ and alpha beta TCR−. CD8+/alpha beta TCR hFCs may be CD8+/alpha beta TCR−/CD56^{dim/neg} or may be CD8+/alpha beta TCR−/CD56^{bright}. hFCs may also be characterized by the presence of cells expressing the following markers: CD3 epsilon CD19, CD11c, CD11b, Foxp3, HLA-DR, and CD123. hFCs may be predominantly CD3 epsilon-/CD19+ cells. For example, about 48% of the hFCs may be CD8+/alpha beta TCR−/CD3 epsilon+ cells. In other aspects of the invention, hFCs may be predominantly CD3 epsilon+/CD19− cells. Among CD8+/alpha beta TCR−/CD56^{dim/neg} cells, the majority of cells express CD3 epsilon. Among CD8+/alpha beta TCR−/CD56^{bright} cells, CD3 epsilon is expressed at a much lower level.

Hematopoietic and human facilitating cells and cellular compositions for engrafting CD3+ cells according to aspects of the invention are described in WIPO PCT Publication Nos. WO/1994/001534; WO/1995/018631; WO/1998/026802; WO/1994/001534; WO/1998/026802; WO/1999/026639; WO/2002/040639; WO/2002/040640; WO/2003/012060; WO/2005/001040; WO/2005/040050; WO/2005/023982; WO/2009/148568; and WO/2018/165161; U.S. Pat. Nos. 5,514,364; 5,635,156; 5,772,994; 5,876,692; 6,039,684; 6,217,867; 7,429,376; 8,632,768; and 9,678,062; and U.S. Patent Publication Nos. US 2001-0009663; US 2002-0142462; US 2002-089746; US 2003-0055223; US 2003-0017152; US 2003-0165475; US 2004-0185043; US 2004-0228845; US 2005-0118142; US 2006-0018885; US 2007-0098693; US 2010-0016240; US 2011-0110909; US 2014-0234843, US 2017-0000825; and US 2018-0252729; each of which is herein incorporated by reference.

Advantageously, the methods of the present invention are effective to reduce the amount, duration, or both the amount and duration of immunosuppressant therapy provided to the recipient after implanting the human organ in the recipient. For example, where an immunosuppressant therapy would have been provided to the recipient following the organ transplant, a reduced amount or no immunosuppressant therapy may be administered owing to the TBI and TLI conditioning regimes disclosed herein. Additionally, because the recipient has received a thorough immunosuppressive treatment, survival, function, and engraftment of CD34+ cells is improved and tolerance established, the duration during which an immunosuppressant therapy may be provided to the recipient will be reduced or eliminated and the number of CD34+ cells required is reduced.

The method of the present invention can be used to improve the survival and function of donor CD34+ and/or CD3+ cells and transplantation of any organ. Methods of the invention may be used in conjunction with transplantation of any organ. The organ may be a solid organ and may be selected from a group consisting of a heart, intestine, liver, lung, pancreas and kidney. Preferably, the solid organ is a kidney. In aspects of the invention the organ is a non-solid organ and may be selected from a group consisting of bone marrow, peripheral blood, and certain lymphoid tissue.

In aspects of the invention, the improved survival and function of the donor CD3+ cells facilitates mixed chimerism in the recipient by facilitating engraftment of the donor CD34+ cells and thereby allowing both donor and host myeloid lineages to exist in the host, followed by tolerization by intrathymic negative selection of the T-cells of the host or donor that would otherwise attach donor tissue or host tissue, respectively.

CD3+ and CD34+ cells may be obtained from one or more apheresis products, which may be from a donor before or after they have already donated an organ. Preferably, the CD34⁺ cells are present in amount greater than 1×10⁴ CD34+ cells/kg recipient, and the CD3+ cells are present in an amount greater than 1×10⁴ CD3+ cells/kg recipient. Although, the skilled artisan will recognize that other CD34+ and CD3+ cell concentrations are within the scope of the invention, as exemplified throughout the application.

For example, the cellular products used herein may include various concentrations for each of the CD34⁺ cells and CD3⁺ cells, and different concentrations are discussed herein. The amount may be specified as a number of cells relative to the body mass of the recipient. For example, the cellular product may contain at least 1×10, 2×10¹, 5×10⁵, 1×10⁶, 2×10⁶, or 4×10⁶ CD34⁺ cells/kg recipient weight. The cellular product may contain at least 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, or 1×10⁸ CD3⁺ cells/kg recipient weight.

Donor derived CD3+ and CD34+ cells may be obtained and/or amplified at any point after the subject has donated an organ. For example, an apheresis product may be obtained at least 1 day, at least 2 days, at least 3 days, at least 4 days, at least 5 days, at least 1 week, at least 2 weeks, at least 4 weeks, at least 8 weeks, at least 12 weeks, at least 24 weeks, at least 1 year, at least 2 years, or at least 5 years after the subject has donated an organ.

The solid organ may be any solid or non-solid organ that can be transplanted according to methods known in the art. For example and without limitation, the solid organ may be a kidney, lung, pancreas, pancreatic, islet cells, heart, intestine, colon, liver, skin, muscle, gum, eye, or tooth. Preferably, the solid organ is a kidney. In other aspects, the non-solid organ is bone marrow, peripheral blood, and certain lymphoid tissue. In certain embodiments, the invention is useful in treating brain or hematologic disorders. In aspects of the invention, CD34+ cells may produce a lineage of microglial useful for the treatment of certain Central Nervous System diseases, such as brain disorders. In aspects of the invention, CD34+ cells may produce a lineage of normal blood cells useful for the treatment of beta thalassemia or sickle cell anemia.

The CD3⁺ cells, as well as the CD34+ cells may be HLA-matched to the recipient. The CD3⁺ cells and/or the CD34⁺ cells may be HLA-mismatched to the recipient. The donor and recipient may be HLA-matched at six, eight, ten, or twelve alleles among the HLA-A, HLA-B, HLA-C, HLA-DP, HLA-DQ, and HLA-DR genes. The donor and recipient may be HLA-mismatched at one, two, three, four, five, six, or more alleles among the HLA-A, HLA-B, HLA-C, HLA-DP, HLA-DQ, and HLA-DR genes.

In addition, the CD3⁺ cells, as well as the CD34+ and/or hFCs may be HLA-matched to the recipient. The CD3⁺ cells, CD34⁺ cells, and/or hFCs may be HLA-mismatched to the recipient. The donor and recipient may be HLA-matched at six, eight, ten, or twelve alleles among the HLA-A, HLA-B, HLA-C, HLA-DP, HLA-DQ, and HLA-DR genes. The donor and recipient may be HLA-mismatched at one, two, three, four, five, six, or more alleles among the HLA-A, HLA-B, HLA-C, HLA-DP, HLA-DQ, and HLA-DR genes.

The cells may be cryopreserved. The cells may contain one or more cryoprotectants. The cryoprotectant may be dextran having an average molecular weight of 40,000 Da or DMSO. The cellular product may contain the cryoprotectant at a concentration of about 1%, 2%, 3%, 4%, 5%, 7.5%, or 10%. The cells may be provided in separate containers. The cells may be provided as a mixture in the same container.

### DETAILED DESCRIPTION

The primary hurdle in organ transplantation is getting the recipient to tolerate the donor's tissue. If the recipient's immune system detects the donated organ as foreign, it attacks the tissue, leading to graft rejection. Consequently, most transplant recipients must take a combination of drugs that suppress the immune system. Immunosuppressive therapy, however, creates its own set of risks. To avoid graft rejection, transplantation of organs may be accompanied by transfer of donor derived CD3+ cells and CD34+ cells. Providing donor blood cells allows reconstitution of the recipient's immune system to include cells that have been educated to recognize the organ as non-foreign tissue. Consequently, the donated organ is not attacked, and the recipient tolerates the graft.

One strategy for reconstructing the recipient's immune system entails repopulating the recipient's immune system with donor-derived CD34+ cells. Successful engraftment of CD34+ cells allows for complete discontinuation of immunosuppression after the CD34+ cells are successfully engrafted.

However, in order to facilitate engraftment of the donor-derived blood cell progenitors and the organ transplant itself, recipients are often conditioned with an immunosuppressive regime that includes doses of irradiation.

The present invention provides methods used to condition recipients of organ and tissue transplants to achieve tolerance, and eventually achieve persistent mixed donor cell chimerism, using a novel non-myeloablative conditioning regime featuring total lymphoid irradiation (TLI) and total body irradiation (TBI), each provided as a fractionated series of doses. Advantageously, this regime not only provides better conditioning than existing non-myeloablative regimes, but also does not require extending the typical duration of the conditioning course.

The present invention is based on the insight that an immunosuppressive regime including a plurality of fractionated doses of both TLI and TBI suppresses a recipient's immune system to effectively facilitate engraftment, while also avoiding complete eradication of the recipient's immune system, which would prevent establishment of mixed chimerism.

In general, the methods of the invention include transplanting an organ or tissue from a donor into a recipient, performing a non-myeloablative conditioning regime of the invention, and infusing the recipient with a hematopoietic cell composition. The non-myeloablative conditioning regimes of the invention combine a plurality of TLI and TBI doses, i.e., at least two of each TLI and TBI. In particular, the total irradiation provided by the multiple TBI doses is far below the amount of irradiation generally used in TBI conditioning regimes—on the order of about between 30 and 150 cGy. Merely providing this low dose of TBI with TLI, as a series of fractionated doses, destroys sufficient numbers of immune cells of a transplant recipient, including in areas of the body not otherwise impacted by TLI administration alone. Areas of the body, particularly organs (e.g., the liver and lungs), not targeted by TLI may harbor fairly large quantities of a recipient's own immune cells—which could attack a transplanted organ or donor-derived CD3+ cells and CD34+ cells used to help establish mixed chimerism. As TBI broadly targets the torso, these remnant sources of immune cells may be sufficiently depleted to encourage engraftment.

The presently disclosed methods may be used in recipients of a solid organ and/or tissue transplant. These methods find applicability in recipients receiving organs from living or deceased donors, including from HLA mismatched donors. The presently claimed methods may lead to persistent mixed chimerism in a recipient, allowing for withdrawal or cessation of immunosuppressive therapies, while concurrently preventing rejection of any transplanted graft.

In some cases, the irradiation is delivered intraoperatively. In some cases, the irradiation is delivered intravenously. In some cases, the irradiation is delivered intraarterially. In some cases, the irradiation is delivered subcutaneously. In some cases, the irradiation is delivered intraperitoneally.

In some cases, a conditioning method of the invention includes a plurality of doses of TBI. For example, a recipient may receive at least two doses of TBI irradiation, three doses of TBI irradiation, four doses of TBI irradiation, five doses of TBI irradiation, six doses of TBI irradiation, seven doses of TBI irradiation, eight doses of TBI irradiation, nine doses of TBI irradiation, 10 doses of TBI irradiation, 11 doses of TBI irradiation, 12 doses of TBI irradiation, 13 doses of TBI irradiation, 14 doses of TBI irradiation, 15 doses of TBI irradiation, 16 doses of TBI irradiation, 17 doses of TBI irradiation, 18 doses of TBI irradiation, 19 doses of TBI irradiation, or at least 20 doses of TBI irradiation.

In some cases, each dose of TBI irradiation may be at least 0.1 cGy, 0.2 cGy, 0.3 cGy, 0.4 cGy, 0.5 cGy, 0.6 cGy, 0.7 cGy, 0.8 cGy, 0.9 cGy, 1 cGy, 2 cGy, 3 cGy, 4 cGy, 5 cGy, 6 cGy, 7 cGy, 8 cGy, 9 cGy, 10 cGy, 11 cGy, 12 cGy, 13 cGy, 14 cGy, 15 cGy, 16 cGy, 17 cGy, 18 cGy, 19 cGy, 20 cGy, 21 cGy, 22 cGy, 23 cGy, 24 cGy, 25 cGy, 26 cGy, 27 cGy, 28 cGy, 29 cGy, 30 cGy, 31 cGy, 32 cGy, 33 cGy, 34 cGy, 35 cGy, 36 cGy, 37 cGy, 38 cGy, 39 cGy, 40 cGy, 41 cGy, 42 cGy, 43 cGy, 44 cGy, 45 cGy, 46 cGy, 47 cGy, 48 cGy, 49 cGy, 50 cGy, 51 cGy, 52 cGy, 53 cGy, 54 cGy, 55 cGy, 56 cGy, 57 cGy, 58 cGy, 59 cGy, 60 cGy, 61 cGy, 62 cGy, 63 cGy, 64 cGy, 65 cGy, 66 cGy, 67 cGy, 68 cGy, 69 cGy, 70 cGy, 71 cGy, 72 cGy, 73 cGy, 74 cGy, 75 cGy, 76 cGy, 77 cGy, 78 cGy, 79 cGy, 80 cGy, 81 cGy, 82 cGy, 83 cGy, 84 cGy, 85 cGy, 86 cGy, 87 cGy, 88 cGy, 89 cGy, 90 cGy, 91 cGy, 92 cGy, 93 cGy, 94 cGy, 95 cGy, 96 cGy, 97 cGy, 98 cGy, 99 cGy, 100 cGy, 105 cGy, 110 cGy, 115 cGy, 120 cGy, 125 cGy, 130 cGy, 135 cGy, 140 cGy, 145 cGy, 150 cGy, 155 cGy, 160 cGy, 165 cGy, 170 cGy, 175 cGy, 180 cGy, 185 cGy, 190 cGy, 195 cGy, 200 cGy, 205 cGy, 210 cGy, 215 cGy, 220 cGy, 225 cGy, 230 cGy, 235 cGy, 240 cGy, 245 cGy, 250 cGy, 255 cGy, 260 cGy, 265 cGy, 270 cGy, 275 cGy, 280 cGy, 285 cGy, 290 cGy, 295 cGy, 300 cGy, 305 cGy, 310 cGy, 315 cGy, 320 cGy, 325 cGy, 330 cGy, 335 cGy, 340 cGy, 345 cGy, 350 cGy, 355 cGy, 360 cGy, 365 cGy, 370 cGy, 375 cGy, 380 cGy, 385 cGy, 390 cGy, 395 cGy, 400 cGy, 405 cGy, 410 cGy, 415 cGy, 420 cGy, 425 cGy, 430 cGy, 435 cGy, 440 cGy, 445 cGy, 450 cGy, 455 cGy, 460 cGy, 465 cGy, 470 cGy, 475 cGy, 480 cGy, 485 cGy, 490 cGy, 495 cGy or at least 500 cGy.

In some cases, in conjunction with the plurality of TBI doses, a method of the invention includes at least one, but preferably, a plurality of doses of TLI. For example, a recipient may receive at least two doses of TLI irradiation, three doses of TLI irradiation, four doses of TLI irradiation, five doses of TLI irradiation, six doses of TLI irradiation, seven doses of TLI irradiation, eight doses of TLI irradiation, nine doses of TLI irradiation, 10 doses of TLI irradiation, 11 doses of TLI irradiation, 12 doses of TLI irradiation, 13 doses of TLI irradiation, 14 doses of TLI irradiation, 15 doses of TLI irradiation, 16 doses of TLI irradiation, 17 doses of TLI irradiation, 18 doses of TLI irradiation, 19 doses of TLI irradiation, or at least 20 doses of TLI irradiation.

In some cases, each dose of TLI irradiation may be at least 0.1 cGy, 0.2 cGy, 0.3 cGy, 0.4 cGy, 0.5 cGy, 0.6 cGy, 0.7 cGy, 0.8 cGy, 0.9 cGy, 1 cGy, 2 cGy, 3 cGy, 4 cGy, 5 cGy, 6 cGy, 7 cGy, 8 cGy, 9 cGy, 10 cGy, 11 cGy, 12 cGy, 13 cGy, 14 cGy, 15 cGy, 16 cGy, 17 cGy, 18 cGy, 19 cGy, 20 cGy, 21 cGy, 22 cGy, 23 cGy, 24 cGy, 25 cGy, 26 cGy, 27 cGy, 28 cGy, 29 cGy, 30 cGy, 31 cGy, 32 cGy, 33 cGy, 34 cGy, 35 cGy, 36 cGy, 37 cGy, 38 cGy, 39 cGy, 40 cGy, 41 cGy, 42 cGy, 43 cGy, 44 cGy, 45 cGy, 46 cGy, 47 cGy, 48 cGy, 49 cGy, 50 cGy, 51 cGy, 52 cGy, 53 cGy, 54 cGy, 55 cGy, 56 cGy, 57 cGy, 58 cGy, 59 cGy, 60 cGy, 61 cGy, 62 cGy, 63 cGy, 64 cGy, 65 cGy, 66 cGy, 67 cGy, 68 cGy, 69 cGy, 70 cGy, 71 cGy, 72 cGy, 73 cGy, 74 cGy, 75 cGy, 76 cGy, 77 cGy, 78 cGy, 79 cGy, 80 cGy, 81 cGy, 82 cGy, 83 cGy, 84 cGy, 85 cGy, 86 cGy, 87 cGy, 88 cGy, 89 cGy, 90 cGy, 91 cGy, 92 cGy, 93 cGy, 94 cGy, 95 cGy, 96 cGy, 97 cGy, 98 cGy, 99 cGy, 100 cGy, 105 cGy, 110 cGy, 115 cGy, 120 cGy, 125 cGy, 130 cGy, 135 cGy, 140 cGy, 145 cGy, 150 cGy, 155 cGy, 160 cGy, 165 cGy, 170 cGy, 175 cGy, 180 cGy, 185 cGy, 190 cGy, 195 cGy, 200 cGy, 205 cGy, 210 cGy, 215 cGy, 220 cGy, 225 cGy, 230 cGy, 235 cGy, 240 cGy, 245 cGy, 250 cGy, 255 cGy, 260 cGy, 265 cGy, 270 cGy, 275 cGy, 280 cGy, 285 cGy, 290 cGy, 295 cGy, 300 cGy, 305 cGy, 310 cGy, 315 cGy, 320 cGy, 325 cGy, 330 cGy, 335 cGy, 340 cGy, 345 cGy, 350 cGy, 355 cGy, 360 cGy, 365 cGy, 370 cGy, 375 cGy, 380 cGy, 385 cGy, 390 cGy, 395 cGy, 400 cGy, 405 cGy, 410 cGy, 415 cGy, 420 cGy, 425 cGy, 430 cGy, 435 cGy, 440 cGy, 445 cGy, 450 cGy, 455 cGy, 460 cGy, 465 cGy, 470 cGy, 475 cGy, 480 cGy, 485 cGy, 490 cGy, 495 cGy or at least 500 cGy.

Irradiation may be administered on the same day of organ transplantation. In some cases, the plurality of irradiation doses may be delivered over a period of time after organ transplantation. In some cases, the plurality of irradiation doses may be delivered over a period of at least 1 day, at least 2 days, at least 1 week, at least 2 week, 3 weeks, or more. In some cases, the doses of irradiation are delivered on a regular interval over the course of administration. In other cases, the doses of irradiation are not delivered on a regular interval over the course of administration. For example, irradiation may be delivered to the thymus gland on days 1 through 4, and days 7 through 11 after transplantation.

In preferred aspects, doses of TBI are administered in the last one or two days of the regime.

In preferred aspects, the total combined irradiation provided by the plurality of TBI doses is between about 30 and about 150 cGy. In even more preferred aspects, the total combined irradiation provided by the plurality of TLI doses is between about 1100 and about 1300 cGy. In preferred aspects, the total combined irradiation provided by the plurality of TLI doses is about 1200 cGy.

The irradiation may be targeted to a particular location of the recipient's body. In some cases, the irradiation may be targeted to a tissue, an organ, a region of the body or the whole body. In some cases, irradiation may be targeted to the lymph nodes, the spleen, or the thymus or any other area known to a person of skill in the art. In some cases, the irradiation may be targeted to the same location when at least more than one dose of irradiation is delivered to the patient. In other cases, the irradiation may be targeted a different location when at least more than one dose of irradiation is delivered to the patient.

During conditioning, recipients may be monitored for the development of conditions associated with non-myeloablative conditioning. Such diseases include neutropenia (e.g., granulocytes<2,000/mL), thrombocytopenia (e.g., platelets<60,000/mL) and secondary infections. In some cases, G-CSF (e.g., 10 gg/kg/day) may be administered for neutropenia. In some cases, any standard treatment known to one of skill in the art may be administered for thrombocytopenia or any secondary infections.

Following transplantation of the HLA-matched or HLA-mismatched organ, the recipient may be treated with non-myeloablative conditioning in addition to the combined TLI and TBI regime. This additional conditioning may include one or more agents. In preferred aspects, the additional conditioning agent is administration of ATG, which is a known T cell depleting agent.

In some cases, ATG may be delivered intravenously. In some cases, a single dose of ATG may be delivered to the recipient. In other cases, the recipient may receive more than one dose of ATG For example, a recipient may receive at least one dose of ATG, two doses of ATG, three doses of ATG, four doses of ATG, five doses of ATG, six doses of ATG, seven doses of ATG, eight doses of ATG, nine doses of ATG, 10 doses of ATG, 11 doses of ATG, 12 doses of ATG, 13 doses of ATG, 14 doses of ATG, 15 doses of ATG, 16 doses of ATG, 17 doses of ATG, 18 doses of ATG, 19 doses of ATG, or at least 20 doses of ATG.

In some cases, each dose of ATG may be at least 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg kg, 4.9 mg/kg, 5.0 mg/kg, 5.1 mg kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg/kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, 10 mg/kg, 10.5 mg/kg, 11 mg/kg, 11.5 mg/kg, 12 mg/kg, 12.5 mg/kg, 13 mg/kg, 13.5 mg/kg, 14 mg/kg, 14.5 mg/kg, 15 mg/kg, 15.5 mg/kg, 16 mg/kg, 16.5 mg/kg, 17 mg/kg, 17.5 mg/kg, 18 mg/kg, 18.5 mg/kg, 19 mg/kg or at least 20 mg/kg.

ATG may be administered on the same day of solid-organ transplantation. In some cases, the plurality of ATG doses may be administered over a period of time after organ transplantation. In some cases, the plurality of ATG doses may be administered over a period of at least 0.1 days, 0.2 days, 0.3 days, 0.4 days, 0.5 days, 0.6 days, 0.7 days, 0.8 days, 0.9 days, 1.0 days, 1.1 days, 1.2 days, 1.3 days, 1.4 days, 1.5 days, 1.6 days, 1.7 days, 1.8 days, 1.9 days, 2.0 days, 2.1 days, 2.2 days, 2.3 days, 2.4 days, 2.5 days, 2.6 days, 2.7 days, 2.8 days, 2.9 days, 3.0 days, 3.1 days, 3.2 days, 3.3 days, 3.4 days, 3.5 days, 3.6 days, 3.7 days, 3.8 days, 3.9 days, 4.0 days, 4.1 days, 4.2 days, 4.3 days, 4.4 days, 4.5 days, 4.6 days, 4.7 days, 4.8 days, 4.9 days, 5.0 days, 5.1 days, 5.2 days, 5.3 days, 5.4 days, 5.5 days, 5.6 days, 5.7 days, 5.8 days, 5.9 days, 6.0 days, 6.1 days, 6.2 days, 6.3 days, 6.4 days, 6.5 days, 6.6 days, 6.7 days, 6.8 days, 6.9 days, 7.0 days, 7.1 days, 7.2 days, 7.3 days, 7.4 days, 7.5 days, 7.6 days, 7.7 days, 7.8 days, 7.9 days, 8.0 days, 8.1 days, 8.2 days, 8.3 days, 8.4 days, 8.5 days, 8.6 days, 8.7 days, 8.8 days, 8.9 days, 9.0 days, 9.1 days, 9.2 days, 9.3 days, 9.4 days, 9.5 days, 9.6 days, 9.7 days, 9.8 days, 9.9 days, 10 days, 10.5 days, 11 days, 11.5 days, 12 days, 12.5 days, 13 days, 13.5 days, 14 days, 14.5 days, 15 days, 15.5 days, 16 days, 16.5 days, 17 days, 17.5 days, 18 days, 18.5 days, 19 days or at least 20 days.

In some cases, the ATG is delivered intra-operatively before the transplanted organ is perfused with host blood. In other cases, the ATG is delivered intra-operatively after the transplanted organ is perfused with host blood. In some cases, the ATG is delivered intravenously before the transplanted organ is perfused with host blood. In other cases, the ATG is delivered intra-venously after the transplanted organ is perfused with host blood. In some cases, the ATG is delivered intra-arterially before the transplanted organ is perfused with host blood. In other cases, the ATG is delivered intra-arterially after the transplanted organ is perfused with host blood. In some cases, the ATG is delivered subcutaneously before the transplanted organ is perfused with host blood. In other cases, the ATG is delivered subcutaneously after the transplanted organ is perfused with host blood. In some cases, the ATG is delivered intraperitoneally before the transplanted organ is perfused with host blood. In other cases, the ATG is delivered intraperitonially after the transplanted organ is perfused with host blood.

Corticosteroid therapy may be given as medication prior to administration of ATG. In some cases, solumedrol may be administered although any corticosteroid known to one of skill in the art sufficient to reduce side effects of ATG may be used at an effective dose. In some cases, the corticosteriod may be administered on the same day as ATG is administered. For example, solumedrol may be administered at a dose within the range of 0-40 mg, 5-50 mg, 10-60 mg, 15-65 mg, 20-70 mg, 25-75 mg, 30-80 mg, 35-85 mg, 40-90 mg, 45-95 mg, 50-100 mg, 55-105 mg, 60-110 mg, 65-115 mg, 70-120 mg, 75-125 mg, 80-130 mg, 85-135 mg, 90-140 mg, 95-145 mg, 100-150 mg, 105-155 mg, 110-160 mg, 115-165 mg, 120-170 mg, 125-175 mg, 130-180 mg, 135-185 mg, 140-190 mg, 145-195 mg or 150-200 mg.

Following the final dose of ATG administered to the recipient, prednisone may be administered. In some cases, a single dose of prednisone may be administered. In other cases, more than one dose of prednisone may be administered. For example, multiple doses of prednisone may be administered according to a tapering course or a constant course.

In some cases, for a tapering course, the first dose of prednisone may start at 100 mg/d and then the dose reduced by 5 mg/d until constant at 5 mg/d for at least 15 days, the first dose of prednisone may start at 90 mg/d and reduced by 5 mg/d until constant for at least 15 days, the first dose of prednisone may start at 80 mg/d and reduced by 5 mg/d until constant for at least 15 days, the first dose of prednisone may start at 70 mg/d and reduced by 5 mg/d until constant for at least 15 days, the first dose of prednisone may start at 60 mg/d and reduced by 5 mg/d until constant for at least 15 days, the first dose of prednisone may start at 50 mg/d and reduced by 5 mg/d until constant for at least 15 days, the first dose of prednisone may start at 40 mg/d and reduced by 5 mg/d until constant for at least 15 days, the first dose of prednisone may start at 30 mg/d and reduced by 5 mg/d until constant for at least 15 days, the first dose of prednisone may start at 20 mg/d and reduced by 5 mg/d until constant for at least 15 days or the first dose of prednisone may start at 10 mg/d and reduced by 5 mg/d until constant for at least 15 days. In some cases, for a constant course, the doses of prednisone may be 100 mg/d, 90 mg/d, 80 mg/d, 70 mg/d, 60 mg/d, 50 mg/d, 40 mg/d, 30 mg/d, 20 mg/d, 10 mg/d or 5 mg/d for at least 15 days.

In some cases, for a tapering course, the first dose of prednisone may start at 100 mg/d and reduced by 5 mg/d until constant for at least 30 days, the first dose of prednisone may start at 90 mg/d and reduced by 5 mg/d until constant for at least 30 days, the first dose of prednisone may start at 80 mg/d and reduced by 5 mg/d until constant for at least 30 days, the first dose of prednisone may start at 70 mg/d and reduced by 5 mg/d until constant for at least 30 days, the first dose of prednisone may start at 60 mg/d and reduced by 5 mg/d until constant for at least 30 days, the first dose of prednisone may start at 50 mg/d and reduced by 5 mg/d until constant for at least 30 days, the first dose of prednisone may start at 40 mg/d and reduced by 5 mg/d until constant for at least 30 days, the first dose of prednisone may start at 30 mg/d and reduced by 5 mg/d until constant for at least 30 days, the first dose of prednisone may start at 20 mg/d and reduced by 5 mg/d until constant for at least 30 days or the first dose of prednisone may start at 10 mg/d and reduced by 5 mg/d until constant for at least 30 days. In some cases, for a constant course, the doses of prednisone may be 100 mg/d, 90 mg/d, 80 mg/d, 70 mg/d, 60 mg/d, 50 mg/d, 40 mg/d, 30 mg/d, 20 mg/d, 10 mg/d or 5 mg/d for at least 30 days.

In some cases, for a tapering course, the first dose of prednisone may start at 100 mg/d and reduced by 5 mg/d until constant for at least 45 days, the first dose of prednisone may start at 90 mg/d and reduced by 5 mg/d until constant for at least 45 days, the first dose of prednisone may start at 80 mg/d and reduced by 5 mg/d until constant for at least 45 days, the first dose of prednisone may start at 70 mg/d and reduced by 5 mg/d until constant for at least 45 days, the first dose of prednisone may start at 60 mg/d and reduced by 5 mg/d until constant for at least 45 days, the first dose of prednisone may start at 50 mg/d and reduced by 5 mg/d until constant for at least 45 days, the first dose of prednisone may start at 40 mg/d and reduced by 5 mg/d until constant for at least 45 days, the first dose of prednisone may start at 30 mg/d and reduced by 5 mg/d until constant for at least 45 days, the first dose of prednisone may start at 20 mg/d and reduced by 5 mg/d until constant for at least 45 days or the first dose of prednisone may start at 10 mg/d and reduced by 5 mg/d until constant for at least 45 days. In some cases, for a constant course, the doses of prednisone may be 100 mg/d, 90 mg/d, 80 mg/d, 70 mg/d, 60 mg/d, 50 mg/d, 40 mg/d, 30 mg/d, 20 mg/d, 10 mg/d or 5 mg/d for at least 45 days.

In some cases, for a tapering course, the first dose of prednisone may start at 100 mg/d and reduced by 5 mg/d until constant for at least 60 days, the first dose of prednisone may start at 90 mg/d and reduced by 5 mg/d until constant for at least 60 days, the first dose of prednisone may start at 80 mg/d and reduced by 5 mg/d until constant for at least 60 days, the first dose of prednisone may start at 70 mg/d and reduced by 5 mg/d until constant for at least 60 days, the first dose of prednisone may start at 60 mg/d and reduced by 5 mg/d until constant for at least 60 days, the first dose of prednisone may start at 50 mg/d and reduced by 5 mg/d until constant for at least 60 days, the first dose of prednisone may start at 40 mg/d and reduced by 5 mg/d until constant for at least 60 days, the first dose of prednisone may start at 30 mg/d and reduced by 5 mg/d until constant for at least 60 days, the first dose of prednisone may start at 20 mg/d and reduced by 5 mg/d until constant for at least 60 days or the first dose of prednisone may start at 10 mg/d and reduced by 5 mg/d until constant for at least 60 days. In some cases, for a constant course, the doses of prednisone may be 100 mg/d, 90 mg/d, 80 mg/d, 70 mg/d, 60 mg/d, 50 mg/d, 40 mg/d, 30 mg/d, 20 mg/d, 10 mg/d or 5 mg/d for at least 60 days.

The corticosteroid and/or prednisone may be administered intravascularly, intravenously, intraarterially, intracranially, intraperitoneally, subcutaneously, intramuscularly, intraorbitally, orally, topically, or through any source which permits proper metabolism of the corticosteroid and/or prednisone by the recipient.

In some cases, any T cell depleting agent known to one of skill in the art can be used as a portion of a non-myeloablative conditioning regime for the recipient. In some cases, the T cell depleting agent may be an anti-T cell monoclonal antibody or a T cell depleting drug (e.g., fludarabine). In some cases, a single T cell depleting agent is administered to the recipient. In other cases, more than one T cell depleting agent is administered to the recipient.

In some cases, a T cell depleting agent may be delivered intravenously. In some cases, a single dose of a T cell depleting agent may be delivered to the recipient. In other cases, the recipient may receive more than one dose of a T cell depleting agent. For example, a recipient may receive at least one dose of a T cell depleting agent, two doses of a T cell depleting agent, three doses of a T cell depleting agent, four doses of a T cell depleting agent, five doses of a T cell depleting agent, six doses of a T cell depleting agent, seven doses of a T cell depleting agent, eight doses of a T cell depleting agent, nine doses of a T cell depleting agent, 10 doses of a T cell depleting agent, 11 doses of a T cell depleting agent, 12 doses of a T cell depleting agent, 13 doses of a T cell depleting agent, 14 doses of a T cell depleting agent, 15 doses of a T cell depleting agent, 16 doses of a T cell depleting agent, 17 doses of a T cell depleting agent, 18 doses of a T cell depleting agent, 19 doses of a T cell depleting agent, or 20 doses of a T cell depleting agent.

A T cell depleting agent may be administered on the same day of solid-organ transplantation. In some cases, the plurality of T cell depleting agent doses may be delivered over a period of time after organ transplantation. In some cases, the plurality of T cell depleting agent doses may be delivered over a period of at least 0.1 days, 0.2 days, 0.3 days, 0.4 days, 0.5 days, 0.6 days, 0.7 days, 0.8 days, 0.9 days, 1.0 days, 1.1 days, 1.2 days, 1.3 days, 1.4 days, 1.5 days, 1.6 days, 1.7 days, 1.8 days, 1.9 days, 2.0 days, 2.1 days, 2.2 days, 2.3 days, 2.4 days, 2.5 days, 2.6 days, 2.7 days, 2.8 days, 2.9 days, 3.0 days, 3.1 days, 3.2 days, 3.3 days, 3.4 days, 3.5 days, 3.6 days, 3.7 days, 3.8 days, 3.9 days, 4.0 days, 4.1 days, 4.2 days, 4.3 days, 4.4 days, 4.5 days, 4.6 days, 4.7 days, 4.8 days, 4.9 days, 5.0 days, 5.1 days, 5.2 days, 5.3 days, 5.4 days, 5.5 days, 5.6 days, 5.7 days, 5.8 days, 5.9 days, 6.0 days, 6.1 days, 6.2 days, 6.3 days, 6.4 days, 6.5 days, 6.6 days, 6.7 days, 6.8 days, 6.9 days, 7.0 days, 7.1 days, 7.2 days, 7.3 days, 7.4 days, 7.5 days, 7.6 days, 7.7 days, 7.8 days, 7.9 days, 8.0 days, 8.1 days, 8.2 days, 8.3 days, 8.4 days, 8.5 days, 8.6 days, 8.7 days, 8.8 days, 8.9 days, 9.0 days, 9.1 days, 9.2 days, 9.3 days, 9.4 days, 9.5 days, 9.6 days, 9.7 days, 9.8 days, 9.9 days, 10 days, 10.5 days, 11 days, 11.5 days, 12 days, 12.5 days, 13 days, 13.5 days, 14 days, 14.5 days, 15 days, 15.5 days, 16 days, 16.5 days, 17 days, 17.5 days, 18 days, 18.5 days, 19 days or at least 20 days.

In some cases, the T cell depleting agent is delivered intra-operatively before the transplanted organ is perfused with host blood. In other cases, the T cell depleting agent is delivered intra-operatively after the transplanted organ is perfused with host blood. In some cases, the T cell depleting agent is delivered intravenously before the transplanted organ is perfused with host blood. In other cases, the T cell depleting agent is delivered intravenously after the transplanted organ is perfused with host blood. In some cases, the T cell depleting agent is delivered intra-arterially before the transplanted organ is perfused with host blood. In other cases, the T cell depleting agent is delivered intra-arterially after the transplanted organ is perfused with host blood. In some cases, the T cell depleting agent is delivered subcutaneously before the transplanted organ is perfused with host blood. In other cases, the T cell depleting agent is delivered subcutaneously after the transplanted organ is perfused with host blood. In some cases, the T cell depleting agent is delivered intraperitoneally before the transplanted organ is perfused with host blood. In other cases, the T cell depleting agent is delivered intraperitoneally after the transplanted organ is perfused with host blood.

In some cases, fludarabine may be delivered intravenously. In some cases, a single dose of fludarabine may be delivered to the recipient. In other cases, the recipient may receive more than one dose of fludarabine. For example, a recipient may receive at least one dose of fludarabine, two doses of fludarabine, three doses of fludarabine, four doses of fludarabine, five doses of fludarabine, six doses of fludarabine, seven doses of fludarabine, eight doses of fludarabine, nine doses of fludarabine, 10 doses of fludarabine, 11 doses of fludarabine, 12 doses of fludarabine, 13 doses of fludarabine, 14 doses of fludarabine, 15 doses of fludarabine, 16 doses of fludarabine, 17 doses of fludarabine, 18 doses of fludarabine, 19 doses of fludarabine, or at least 20 doses of fludarabine.

In some cases, each dose of fludarabine may be at least 0.1 mg/m2/d, 0.2 mg/m2/d, 0.3 mg/m2/d, 0.4 mg/m2/d, 0.5 mg/m2/d, 0.6 mg/m2/d, 0.7 mg/m2/d, 0.8 mg/m2/d, 0.9 mg/m2/d, 1.0 mg/m2/d, 11 mg/m2/d, 1.2 mg/m2/d, 1.3 mg/m2/d, 1.4 mg/m2/d, 1.5 mg/m2/d, 1.6 mg/m2/d, 1.7 mg/m2/d, 1.8 mg/m2/d, 1.9 mg/m2/d, 2.0 mg/m2/d, 2.1 mg/m2/d, 2.2 mg/m2/d, 2.3 mg/m2/d, 2.4 mg/m2/d, 2.5 mg/m2/d, 2.6 mg/m2/d, 2.7 mg/m2/d, 2.8 mg/m2/d, 2.9 mg/m2/d, 3.0 mg/m2/d, 3.1 mg/m2/d, 3.2 mg/m2/d, 3.3 mg/m2/d, 3.4 mg/m2/d, 3.5 mg/m2/d, 3.6 mg/m2/d, 3.7 mg/m2/d, 3.8 mg/m2/d, 3.9 mg/m2/d, 4.0 mg/m2/d, 4.1 mg/m2/d, 4.2 mg/m2/d, 4.3 mg/m2/d, 4.4 mg/m2/d, 4.5 mg/m2/d, 4.6 mg/m2/d, 4.7 mg/m2/d, 4.8 mg/m2/d, 4.9 mg/m2/d, 5.0 mg/m2/d, 5.1 mg/m2/d, 5.2 mg/m2/d, 5.3 mg/m2/d, 5.4 mg/m2/d, 5.5 mg/m2/d, 5.6 mg/m2/d, 5.7 mg/m2/d, 5.8 mg/m2/d, 5.9 mg/m2/d, 6.0 mg/m2/d, 6.1 mg/m2/d, 6.2 mg/m2/d, 6.3 mg/m2/d, 6.4 mg/m2/d, 6.5 mg/m2/d, 6.6 mg/m2/d, 6.7 mg/m2/d, 6.8 mg/m2/d, 6.9 mg/m2/d, 7.0 mg/m2/d, 7.1 mg/m2/d, 7.2 mg/m2/d, 7.3 mg/m2/d, 7.4 mg/m2/d, 7.5 mg/m2/d, 7.6 mg/m2/d, 7.7 mg/m2/d, 7.8 mg/m2/d, 7.9 mg/m2/d, 8.0 mg/m2/d, 8.1 mg/m2/d, 8.2 mg/m2/d, 8.3 mg/m2/d, 8.4 mg/m2/d, 8.5 mg/m2/d, 8.6 mg/m2/d, 8.7 mg/m2/d, 8.8 mg/m2/d, 8.9 mg/m2/d, 9.0 mg/m2/d, 9.1 mg/m2/d, 9.2 mg/m2/d, 9.3 mg/m2/d, 9.4 mg/m2/d, 9.5 mg/m2/d, 9.6 mg/m2/d, 9.7 mg/m2/d, 9.8 mg/m2/d, 9.9 mg/m2/d, 10 mg/m2/d, 10.5 mg/m2/d, 11 mg/m2/d, 11.5 mg/m2/d, 12 mg/m2/d, 12.5 mg/m2/d, 13 mg/m2/d, 13.5 mg/m2/d, 14 mg/m2/d, 14.5 mg/m2/d, 15 mg/m2/d, 15.5 mg/m2/d, 16 mg/m2/d, 16.5 mg/m2/d, 17 mg/m2/d, 17.5 mg/m2/d, 18 mg/m2/d, 18.5 mg/m2/d, 19 mg/m2/d, 20 mg/m2/d, 20.5 mg/m2/d, 21 mg/m2/d, 21.5 mg/m2/d, 22 mg/m2/d, 22.5 mg/m2/d, 23 mg/m2/d, 23.5 mg/m2/d, 24 mg/m2/d, 24.5 mg/m2/d, 25 mg/m2/d, 25.5 mg/m2/d, 26 mg/m2/d, 26.5 mg/m2/d, 27 mg/m2/d, 27.5 mg/m2/d, 28 mg/m2/d, 28.5 mg/m2/d, 29 mg/m2/d, 30 mg/m2/d, 30.5 mg/m2/d, 31 mg/m2/d, 31.5 mg/m2/d, 32 mg/m2/d, 32.5 mg/m2/d, 33 mg/m2/d, 33.5 mg/m2/d, 34 mg/m2/d, 34.5 mg/m2/d, 35 mg/m2/d, 35.5 mg/m2/d, 36 mg/m2/d, 36.5 mg/m2/d, 37 mg/m2/d, 37.5 mg/m2/d, 38 mg/m2/d, 38.5 mg/m2/d, 39 mg/m2/d, 40 mg/m2/d, 40.5 mg/m2/d, 41 mg/m2/d, 41.5 mg/m2/d, 42 mg/m2/d, 42.5 mg/m2/d, 43 mg/m2/d, 43.5 mg/m2/d, 44 mg/m2/d, 44.5 mg/m2/d, 45 mg/m2/d, 45.5 mg/m2/d, 46 mg/m2/d, 46.5 mg/m2/d, 47 mg/m2/d, 47.5 mg/m2/d, 48 mg/m2/d, 48.5 mg/m2/d, 49 mg/m2/d, 50 mg/m2/d, 50.5 mg/m2/d, 51 mg/m2/d, 51.5 mg/m2/d, 52 mg/m2/d, 52.5 mg/m2/d, 53 mg/m2/d, 53.5 mg/m2/d, 54 mg/m2/d, 54.5 mg/m2/d, 55 mg/m2/d, 55.5 mg/m2/d, 56 mg/m2/d, 56.5 mg/m2/d, 57 mg/m2/d, 57.5 mg/m2/d, 58 mg/m2/d, 58.5 mg/m2/d, 59 mg/m2/d or at least 60 mg/m2/d.

Fludarabine may be administered on the same day of solid-organ transplantation. In some cases, the plurality of fludarabine doses may be delivered over a period of time after organ transplantation. In some cases, the plurality of fludarabine doses may be delivered over a period of at least 0.1 days, 0.2 days, 0.3 days, 0.4 days, 0.5 days, 0.6 days, 0.7 days, 0.8 days, 0.9 days, 1.0 days, 1.1 days, 1.2 days, 1.3 days, 1.4 days, 1.5 days, 1.6 days, 1.7 days, 1.8 days, 1.9 days, 2.0 days, 2.1 days, 2.2 days, 2.3 days, 2.4 days, 2.5 days, 2.6 days, 2.7 days, 2.8 days, 2.9 days, 3.0 days, 3.1 days, 3.2 days, 3.3 days, 3.4 days, 3.5 days, 3.6 days, 3.7 days, 3.8 days, 3.9 days, 4.0 days, 4.1 days, 4.2 days, 4.3 days, 4.4 days, 4.5 days, 4.6 days, 4.7 days, 4.8 days, 4.9 days, 5.0 days, 5.1 days, 5.2 days, 5.3 days, 5.4 days, 5.5 days, 5.6 days, 5.7 days, 5.8 days, 5.9 days, 6.0 days, 6.1 days, 6.2 days, 6.3 days, 6.4 days, 6.5 days, 6.6 days, 6.7 days, 6.8 days, 6.9 days, 7.0 days, 7.1 days, 7.2 days, 7.3 days, 7.4 days, 7.5 days, 7.6 days, 7.7 days, 7.8 days, 7.9 days, 8.0 days, 8.1 days, 8.2 days, 8.3 days, 8.4 days, 8.5 days, 8.6 days, 8.7 days, 8.8 days, 8.9 days, 9.0 days, 9.1 days, 9.2 days, 9.3 days, 9.4 days, 9.5 days, 9.6 days, 9.7 days, 9.8 days, 9.9 days, 10 days, 10.5 days, 11 days, 11.5 days, 12 days, 12.5 days, 13 days, 13.5 days, 14 days, 14.5 days, 15 days, 15.5 days, 16 days, 16.5 days, 17 days, 17.5 days, 18 days, 18.5 days, 19 days or at least 20 days.

In some cases, the Fludarabine is delivered intra-operatively before the transplanted organ is perfused with host blood. In other cases, the Fludarabine is delivered intra-operatively after the transplanted organ is perfused with host blood. In some cases, the Fludarabine is delivered intra-venously before the transplanted organ is perfused with host blood. In other cases, the Fludarabine is delivered intra-venously after the transplanted organ is perfused with host blood. In some cases, the Fludarabine is delivered intra-arterially before the transplanted organ is perfused with host blood. In other cases, the Fludarabine is delivered intra-arterially after the transplanted organ is perfused with host blood. In some cases, the Fludarabine is delivered subcutaneously before the transplanted organ is perfused with host blood. In other cases, the Fludarabine is delivered subcutaneously after the transplanted organ is perfused with host blood. In some cases, the Fludarabine is delivered intraperitoneally before the transplanted organ is perfused with host blood. In other cases, the Fludarabine is delivered intraperitoneally after the transplanted organ is perfused with host blood.

Cellular Products

Methods of the invention are useful, among other contexts, for the combined transplantation of a solid organ and hematopoietic cells to a recipient, where tolerance to the graft is established through development of a stable mixed chimera. An individual with stable mixed chimerism, usually for a period of at least six months, is able to withdraw from the use of immunosuppressive drugs after a period of time sufficient to establish tolerance. The methods used the combined plurality of both TBI and TLI irradiation doses to prepare a transplant recipient for administration of a cellular product comprising hematopoietic stem cells derived from a donor.

All blood cells, including the cells of the immune system, are derived from hematopoietic stem cells (HSCs). HSCs are multipotent cells that can differentiate into various specialized cells and also reproduce to generate new HSCs. HSCs that differentiate form either lymphoid progenitors or myeloid progenitors. Lymphoid progenitors give rise to lymphocytes and natural killer cells. Myeloid progenitors produce cells of the myeloid and erythroid lineages, such as erythrocytes, platelets, basophils, neutrophils, eosinophils, monocytes, macrophages, and antigen-presenting cells, such as dendritic cells. In adults, most hematopoietic development occurs in the bone marrow, although maturation and activation of some lymphoid cells occurs in the spleen, thymus, and lymph nodes.

One strategy for reconstructing the recipient's immune system entails complete replacement of the recipient's hematopoietic system with exclusively donor-derived cells to achieve a state of full chimerism. A risk associated with full chimerism, however, is that the completely donor-derived immune system may identify the recipient's tissue as foreign and attack it, a condition called graft-versus-host disease (GVHD). See, e.g., Sach et al., Induction of Tolerance through Mixed Chimerism, Cold Spring Harb Perspect Med 2014; 4:a015529, doi: 10.1101/cshperspect.a015529, the contents of which are incorporated herein by reference. As a result, fully chimeric patients must remain on immunosuppressive therapy indefinitely.

Another strategy is to repopulate the recipient's immune system with a mixture of donor-derived cells and recipient-derived cells to attain a state called mixed chimerism. Compared to full chimerism, mixed chimerism is associated with lower rates of GVHD. In addition, mixed chimeric regimens require lower doses of immunosuppressive therapy initially and allow complete discontinuation of immunosuppression after the stability of the recipient's mixed chimerism has been established. To date, induction of mixed chimerism is the only method of producing graft tolerance in humans without maintaining immunosuppressive therapy.

Current methods of establishing mixed chimerism require transfer of two different populations of hematopoietic cells from the donor. Mature T cells recognize the transplanted organ as “self” tissue and prevent the immune system from attacking it. Mature T cells express the cell-surface marker CD3, and different sub-populations may be found in the blood and lymph nodes. Although CD3⁺ T cells promote tolerance upon transfer, they have a finite lifespan and are unable to regenerate themselves. Consequently, transfer of hematopoietic stem and progenitor cells (HSPCs), pluripotent cells that can differentiate into T cells, is also necessary to allow continual replenishment of the donor-derived T cell population. HSPCs express the cell-surface marker CD34 and reside primarily in the bone marrow. The cellular compositions used in the methods of the invention may include two populations of cells that allow donor HSPCs to develop into mature cells of the immune system in the recipient's body. One population includes CD34⁺ cells. CD34 is a cell surface marker that is expressed in stem cells and their immediate descendants, multipotent progenitor cells, which have not committed to either the myeloid or lymphoid lineage. Consequently, CD34 expression is a useful measure for identifying populations of cells that contain HSPCs. In adults, CD34⁺ cells reside predominantly in the bone marrow.

The cellular compositions used in methods of the invention may also include CD3⁺ cells. CD3 comprises a group of polypeptides that interact with the two polypeptide chains of the T cell receptor to form the T cell receptor complex. The CD3 complex includes a gamma chain, delta chain, and two epsilon chains. CD3 is expressed on the surface of mature T cells and is thus useful as a marker for T cells. CD3⁺ cells are abundant in the circulating blood.

To promote establishment of mixed chimerism in the recipient, the cellular products include CD34⁺ cells and CD3⁺ cells in appropriate quantities. The cellular products may contain CD34⁺ cells and CD3⁺ cells in defined amounts. A useful unit of cell quantity in a product is the number of cells relative to the body mass of the recipient. For example and without limitation, the cellular product may contain at least 1×10⁴, 2×10⁴, 5×10⁴, 1×10⁵, 2×10⁵, 5×10⁵, 1×10⁶, 2×10⁶, or 4×10⁶, 1×10⁷, 2×10⁷, 4×10⁷, 1×10⁸, 2×10⁸, or 5×10⁸ CD34⁺ cells/kg recipient weight. For example and without limitation, the cellular product may contain at least 1×10⁴, 2×10⁴, 5×10⁴, 1×10⁵, 2×10⁵, 5×10⁵, 1×10⁶, 2×10⁶, 5×10⁶, 1×10⁷, 2×10⁷, 5×10⁷, 1×10⁸ CD3⁺, 2×10⁸, or 5×10⁸ cells/kg recipient weight.

The cellular product may contain at least 1×10⁵ CD34⁺ cells/kg recipient weight, at least 2×10⁵ CD34⁺ cells/kg recipient weight, at least 4×10⁵ CD34⁺ cells/kg recipient weight, at least 5×10⁵ CD34⁺ cells/kg recipient weight, at least 1×10⁶ CD34⁺ cells/kg recipient weight, at least 2×10⁶ CD34⁺ cells/kg recipient weight, at least 4×10⁶ CD34⁺ cells/kg recipient weight, at least 5×10⁶ CD34⁺ cells/kg recipient weight, at least 1×10⁷ CD34⁺ cells/kg recipient weight, at least 2×10⁷ CD34⁺ cells/kg recipient weight, at least 4×10⁷ CD34⁺ cells/kg recipient weight, at least 1×10⁸ CD34⁺ cells/kg recipient weight, at least 2×10⁸ CD34⁺ cells/kg recipient weight, at least 4×10⁵ CD34⁺ cells/kg recipient weight, or at least 5×10⁸ CD34⁺ cells/kg recipient weight. The cellular product may contain at least 1×10⁵ CD3⁺ cells/kg recipient weight, at least 2×10⁵ CD3⁺ cells/kg recipient weight, at least 4×10⁵ CD3⁺ cells/kg recipient weight, at least 5×10⁵ CD3⁺ cells/kg recipient weight, at least 1×10⁶ CD3⁺ cells/kg recipient weight, at least 2×10⁶ CD3⁺ cells/kg recipient weight, at least 4×10⁶ CD3⁺ cells/kg recipient weight, at least 5×10⁶ CD3⁺ cells/kg recipient weight, at least 1×10⁷ CD3⁺ cells/kg recipient weight, at least 2×10⁷ CD3⁺ cells/kg recipient weight, at least 4×10⁷ CD3⁺ cells/kg recipient weight, at least 1×10⁸ CD3⁺ cells/kg recipient weight, at least 2×10⁸ CD3⁺ cells/kg recipient weight, at least 4×10⁵ CD3⁺ cells/kg recipient weight, or at least 5×10⁸ CD3⁺ cells/kg recipient weight. The cellular product may contain about 1×10⁵ CD3⁺ cells/kg recipient weight, about 2×10⁵ CD3⁺ cells/kg recipient weight, about 4×10⁵ CD3⁺ cells/kg recipient weight, about 5×10⁵ CD3⁺ cells/kg recipient weight, about 1×10⁶ CD3⁺ cells/kg recipient weight, about 2×10⁶ CD3⁺ cells/kg recipient weight, about 4×10⁶ CD3⁺ cells/kg recipient weight, about 5×10⁶ CD3⁺ cells/kg recipient weight, about 1×10⁷ CD3⁺ cells/kg recipient weight, about 2×10⁷ CD3⁺ cells/kg recipient weight, about 4×10⁷ CD3⁺ cells/kg recipient weight, about 1×10⁸ CD3⁺ cells/kg recipient weight, about 2×10⁸ CD3⁺ cells/kg recipient weight, about 4×10⁸ CD3⁺ cells/kg recipient weight, or about 5×10⁸ CD3⁺ cells/kg recipient weight.

Other concentrations are exemplified in U.S. Pat. Nos. 9,504,717 and 9,561,253, the contents of each of which are incorporated by reference herein in its entirety.

However, the more complete immunosuppression conferred by the conditioning regimes of the invention allow for the use of fewer CD34+ cells relative to prior methods. Accordingly, the present invention includes cellular products administered to recipients who undergo a plurality of TBI and TLI doses.

The cellular product may contain CD34⁺ cells at a designated level of purity. For example, the cellular product may contain CD34⁺ cells that are at least 30%, at least 40%, at least 50%, at least 60%, at least 70%, at least 80%, at least 90%, at least 95%, at least 98%, or at least 99% pure. Other purities are exemplified in U.S. Pat. Nos. 9,504,717 and 9,561,253, the contents of each of which are incorporated by reference herein in its entirety.

The CD34⁺ cells and CD3⁺ cells may be provided as a mixture in one or more containers. The CD34⁺ cells and CD3⁺ cells may be provided in separate containers. Any commercially available container approved to hold cellar products may be used.

The cellular product may be provided frozen. Consequently, the cellular product may contain a cryoprotectant. Any cryoprotectant known in the art may be used. For example and without limitation, the cryoprotectant may be DMSO, dextran having an average molecular weight of 40 kDa, serum, e.g., bovine serum, albumin, e.g., human serum albumin, or cell culture medium. The cryoprotectant may be present at a defined concentration. For example, the cellular product may contain about 1% DMSO, about 2% DMSO, about 5% DMSO, about 7.5% DMSO, about 10% DMSO, about 12.5% DMSO, about 15% DMSO, or about 20% DMSO. The cellular product may contain about 1% dextran, about 2% dextran, about 5% dextran, about 7.5% dextran, about 10% dextran, about 12.5% dextran, about 15% dextran, or about 20% dextran. The cryoprotectant may be a commercially available freezing medium, such as the medium sold under the trade name CryoStor 10 by BioLife Solutions (Bothell, WA). Cryoprotection is discussed in U.S. Pat. Nos. 9,504,717 and 9,561,253, the contents of each of which are incorporated by reference herein in its entirety.

The cellular product may contain agents that enhance engraftment or functional mobilization of the hematopoietic cells in the recipient. The cellular product may contain agents that prevent a negative reaction of the recipient to the hematopoietic cells. For example and without limitation, the pharmaceutical composition may contain a cytokine, chemokine, growth factor, enzyme, excipient, carrier, antibody or a fragment thereof, small molecule, drug, agonist, antagonist, matrix protein, or complementary cell type.

In certain embodiments, the cellular product contains an enzyme, substrate, or both. For example, the cellular products may contain one or more alpha 1,3-fucosyltransferases, a fucose donor, or both. Fucosylation of HSPCs enhances binding to E-selectin and P-selectin and improves their ability to home to bone marrow. Examples of alpha 1,3-fucosyltransferase include alpha 1,3-fucosyltransferase IV, alpha 1,3-fucosyltransferase VI, and alpha 1,3 fucosyltransferase VII. The fucose donor may be GDP-fucose. Fucosylation of HSPCs is described in detail in U.S. Pat. No. 7,776,591, the contents of which are incorporated herein by reference.

The cellular product may contain a buffer. The cellular product may be buffer to maintain physiologically compatible pH. For example, the cellular product may be buffered to a neutral pH, such as from about 6.0 to about 8.0.

The cellular product may be supplied in the form of a pharmaceutical composition, comprising an isotonic excipient prepared under sufficiently sterile conditions for human administration. Choice of the cellular excipient and any accompanying elements of the composition is adapted in accordance with the route and device used for administration. For general principles in medicinal formulation, see Cell Therapy: Stem Cell Transplantation, Gene Therapy, and Cellular Immunotherapy, by G. Morstyn & W. Sheridan. eds., Cambridge University Press, 1996; and Hematopoietic Stem Cell Therapy, E. D. Ball, J. Lister & P. Law, Churchill Livingstone, 2000.

The cellular products may include CD3+ positive cells and CD34+ cells. CD3+ and CD34+ cells may also be administered together with hematopoietic or human facilitating cells (hCFs). CD3+ cells and CD34+ cells are two populations of cells that allow donor HSCs to develop into mature cells of the immune system in the recipient's body.

To promote engraftment of the CD34+ cells, cellular compositions are provided that include CD34+ cells and CD3+ cells in appropriate quantities. Compositions may also be provided that include hCFs in appropriate quantities. For example, an ample supply of CD3⁺ cells may promote engraftment of the CD34+ cells. It is also by the present invention understood that the administration of a calcineurin inhibitor improves survival and function of CD3+ cell, thereby further improving engraftment of CD34+ cells.

The cells may be administered together with agents in addition to calcineurin inhibitors that enhance engraftment of the CD3+ cells. For example, agents may be administered that prevent a negative reaction of the recipient to the hematopoietic cells. For example and without limitation, the pharmaceutical composition may contain a cytokine, chemokine, growth factor, excipient, carrier, antibody or a fragment thereof, small molecule, drug, agonist, antagonist, matrix protein, or complementary cell type.

CD34+ cells are relatively scarce, making up only about 0.1-0.2% of peripheral blood cells in normal, untreated patients. Therefore, the cellular products may contain CD34+ cells that have been purified from an apheresis product to obtain a sufficient number of such cells. For example, the CD34+ cells may be purified using an immunomagnetic column system, as described below. In contrast, CD3+ cells are abundant, accounting for a majority of mononuclear cells in the peripheral blood. Thus, the population of CD3+ cells in the cellular products may be obtained from a portion of the apheresis product that has not been subjected to a column purification step. Alternatively or additionally, CD3+ cells may be obtained from a residual fraction following purification of CD34+ cells, such as the effluent of a column used to purify CD34+ cells.

CD3+ cells and CD34+ cells, and hFCs may be administered defined amounts. Additionally, the hFCs and CD3⁺ cells may be derived from any subject that has donated an organ. The hFCs, CD34⁺ cells, and/or CD3⁺ cells may be from the same subject. The hFCs, CD34⁺ cells, and CD3⁺ cells may be from different subjects. Preferably, the hFCs, CD34⁺ cells, and/or CD3⁺ cells are derived from the subject that donated the organ that has been transplanted into the recipient.

The CD34⁺ cells, CD3⁺ cells, and/or hFCs may be HLA-matched or HLA-mismatched to the recipient. Human leukocyte antigens (HLAs), also called major histocompatibility complex (MHC) antigens, are protein molecules expressed on the surface of cells that confer a unique antigenic identity to these cells. MHC/HLA antigens are target molecules that are recognized by T-cells and natural killer (NK) cells as being derived from the same source of hematopoietic stem cells as the immune effector cells (“self”) or as being derived from another source of hematopoietic reconstituting cells (“non-self”). Two main classes of HLA antigens are recognized: HLA class I and HLA class II. HLA class I antigens (A, B, and C in humans) render each cell recognizable as “self,” whereas HLA class II antigens (DR, DP, and DQ in humans) are involved in reactions between lymphocytes and antigen presenting cells.

A key aspect of the HLA gene system is its polymorphism. Each gene exists in different alleles. Allelic gene products differ in one or more amino acids in the alpha and/or beta domain(s). An individual has two alleles of each gene, for a total of twelve alleles among the HLA-A, HLA-B, HLA-C, HLA-DP, HLA-DQ, and HLA-DR genes. An HLA-matched donor may have a match with the recipient at six, eight, ten, or twelve alleles selected from any combination of the HLA-A, HLA-B, HLA-C, HLA-DP, HLA-DQ, and HLA-DR genes. The genes most important for HLA typing are HLA-A, HLA-B, and HLA-DR, so the donor and recipient may be matched at all six alleles of the HLA-A, HLA-B, and HLA-DR genes. An HLA-mismatched donor may have a mismatch at one, two, three, four, five, six, or more alleles among the HLA-A, HLA-B, HLA-C, HLA-DP, HLA-DQ, and HLA-DR genes. HLA typing may be performed by any method known in the art. Examples of HLA typing methods include serological cytotoxicity, flow cytometry, and DNA typing. Such methods are described in, for example, U.S. Pat. No. 9,561,253, the contents of which are incorporated herein by reference.

The HLA genes are clustered in a super-locus present on chromosome position 6p21. Consequently, the set of alleles present on a single chromosome, i.e., a haplotype, tends to be inherited as a group. Identifying a patient's haplotypes can help predict the probability of finding matching donors and assist in developing a search strategy. Haplotypes vary in how common they are among the general population and in their frequency within different racial and ethnic groups.

Numerous exemplary embodiments are now described below, both HLA matched and HLA mismatched. The skilled artisan will recognize that the below embodiments are exemplary and non-limiting, particularly, the below embodiments do not limit any other part or exemplified cell amounts or combinations in any other part of this application.

Preparation of Cellular Products

As indicated above, CD34⁺ cells make up a low percentage of peripheral blood cells in normal subjects. However, the fraction of CD34⁺ cells in blood can be increased by administering to the subject a factor, such as granulocyte colony stimulating factor (G-CSF), that mobilizes CD34⁺ cells from bone marrow and other sources. Thus, prior to apheresis, the subject may be given G-CSF to mobilize CD34⁺ cells. Regimens for administering G-CSF to a subject prior to apheresis, including the dosage, frequency, and timing of administration, are known in the art and described in, for example, U.S. Pat. No. 9,561,253, the contents of which are incorporated herein by reference.

During preparation of the cellular products of the invention, cells may be frozen at any stage. For example, cells may be frozen immediately after an apheresis product is isolated from a donor but prior to separation into portions, after separation into portions, after purification or enrichment of CD34⁺ cells, or after combining purified CD34⁺ cells with CD3⁺ cells.

Cryopreservation of compositions of the invention may include addition of a cryoprotectant, such as a cryoprotectant described above. Cryopreservation typically involves reducing the temperature of the cell-containing sample at a controlled rate. Cryopreservation may include thawing the cell-containing sample and washing the sample to remove one or more cryoprotectants. Methods and reagents for cryopreservation, including freezing, thawing, and washing samples, are known in the art and described in, for example, U.S. Pat. No. 9,561,253, the contents of which are incorporated herein by reference.

CD34⁺ cells may be purified based on qualitative or quantitative expression of one or more cell surface markers. Examples of suitable cell surface markers include CD34, Thy-1, CD38, and AC133. CD34⁺ cells may be purified based on the presence or absence of a marker or on the level of expression of a marker, e.g., high vs. low.

CD34⁺ cells may be purified by selectively binding a suitable affinity reagent to CD34 or another marker. The affinity reagent may be an antibody, a full-length antibody, a fragment of an antibody, a naturally occurring antibody, a synthetic antibody, an engineered antibody, a full-length affibody, a fragment of an affibody, a full-length affilin, a fragment of an affilin, a full-length anticalin, a fragment of an anticalin, a full-length avimer, a fragment of an avimer, a full-length DARPin, a fragment of a DARPin, a full-length fynomer, a fragment of a fynomer, a full-length kunitz domain peptide, a fragment of a kunitz domain peptide, a full-length monobody, a fragment of a monobody, a peptide, a polyaminoacid, or the like. The affinity reagent may be directly conjugated to a detection reagent and/or purification reagent. The detection reagent and purification reagent may be the same, or they may be different. For example, the detection reagent and/or purification reagent may be fluorescent, magnetic, or the like. The detection reagent and/or purification reagent may be a magnetic particle for column purification, e.g., an immunomagnetic microsphere.

CD34⁺ cells may be isolated, enriched, or purified by any method. For example, CD34⁺ cells may be isolated, enriched, or purified by column purification, flow cytometery, cell sorting, or immunoadsorption column separation. Preferably, CD34⁺ cells are purified using an immunomagnetic column system, such as those sold under the trade name CliniMACS by Miltenyi Biotec Inc. (Auburn, CA), Methods of affinity purification of hematopoietic cells, including CD34⁺ cells, and analysis of purified populations are described in, for example, U.S. Pat. Nos. 9,561,253; 9,452,184; 10 Ng et al., Isolation of human and mouse hematopoietic stem cells, Methods Mol Biol. (2009) 506:13-21. doi: 10.1007/978-1-59745-409-4_2; and Spohn et al., Automated CD34⁺ cell isolation of peripheral blood stem cell apheresis product, Cytotherapy (2015) October; 17(10):1465-71. doi: 10.1016/j.jcyt.2015.04.005, the contents of each of which are incorporated herein by reference. The methods may include positive selection, negative selection, or both.

CD3⁺ cells may be obtained by dividing one or more apheresis products into two portions, using one portion to purify or enrich CD34⁺ cells, and using the second portion as a source of CD3⁺ cells. Alternatively, CD3⁺ cells may be obtained from a portion from which CD34⁺ cells have been purified, such as the effluent of column used to purify CD34⁺ cells, as described in, for example, U.S. Pat. No. 9,561,253, the contents of which are incorporated herein by reference.

CD34⁺ cells and/or CD3⁺ cells may be expanded ex vivo. Expansion may occur prior to, or subsequent to, freezing. Expansion may include providing one or more growth factors, and it may include culturing cells in the presence of another cell type, e.g., feeder cells. Methods for expanding hematopoietic cells are described in, for example, U.S. Pat. No. 9,561,253, the contents of which are incorporated herein by reference.

Administering Cellular Product

The methods of the invention may include administering cellular compositions including CD3+ and CD34+ cells. The cellular composition may include or be administered with one or more agents that promote engraftment, e.g., a calcineurin inhibitor, in a recipient of an organ transplant. The cellular compositions may be provided by any suitable means. For example and without limitation, the cellular compositions may be delivered to the recipient by injection using a needle, catheter, central line or the like. In some cases, the cells, and/or compositions, may be delivered intravascularly, intravenously, intraarterially, subcutaneously, intramuscularly, directly to the bone, or through any source which permits the hematopoietic cells to home to an appropriate site in the recipient such that the hematopoietic cells persist, regenerate and differentiate in the recipient. The cellular compositions may be provided by infusion. Administration of the cellular compositions may be provided in an inpatient procedure or in an outpatient procedure. An inpatient procedure requires admission to a hospital, and the patient may spend one or more nights in the hospital. An outpatient procedure does not require admission to a hospital and may be performed in a non-hospital setting, such as a clinic, doctor's office, home, or other location.

Methods of the present invention may be used in conjunction with transplantation of any solid or non-solid organ. For example and without limitation, the solid organ may be a kidney, lung, pancreas, pancreatic, islet cells, heart, intestine, colon, liver, skin, muscle, gum, eye, or tooth. In aspects of the invention, without limitation the non-solid organ may be bone marrow, peripheral blood, and lymphoid tissue. The transplant may include a complete organ, a portion of an organ, or cells from a tissue of an organ. The cellular product may be provided prior to, during, or subsequent to the organ transplant. For example and without limitation, the cellular product may be provided one, two, three, four, five, or six days or one, two, three, or four weeks prior to the organ transplant, or it may be provided one, two, three, four, five, or six days or one, two, three, or four weeks after the organ transplant.

To facilitate survival and function of CD3+ cells in the recipient and thereby engrafiment of CD34+ improved by the presence of the CD3+ cells, the recipient's immune system may be conditioned using the combined plurality of TLI and TBI doses in conjunction with providing the cellular product. Typically, the conditioning regimen includes treatment with anti-thymocyte globulin (ATG), total lymphoid irradiation, and corticosteroids (e.g., prednisone) for a period of from about 10 to 12 days (e.g., for about 11 days).

When multiple doses of irradiation are administered, the doses may be targeted to the same location or to different locations. Non-myeloablative conditioning may include the use of a T cell depleting agent, such as a monoclonal antibody or drug, e.g., fludarabine. Regimens for non-myeloablative conditioning are known in the art and are described in, for example, U.S. Pat. No. 9,561,253, the contents of which are incorporated herein by reference.

The methods may include immunosuppressive therapy. Advantageously, because the administration of the combined plurality of TLI and TBI doses increases the survival and efficacy of CD3+ cells in the recipient and thereby engraftment of CD34+ cells, the duration and/or the amount of immunosuppressive therapies can be reduced. Immunosuppressive therapy, or immunosuppression, involves treatment of the graft recipient with agents that diminish the response of the host immune system against the donor cells, which can lead to graft rejection. Because the regimens and compositions of the present invention result in improved survival and function of the donor CD3+ cells, engraftment of donor CD34+ cells is improved and the need for immunosuppressive therapy is greatly reduced. These immunosuppressive therapies primary immunosuppressive agents include antibody-based therapies, such as use monoclonal (e.g., muromonab-CD3) or polyclonal antibodies or anti-CD25 antibodies (e.g., basiliximab, daclizumab). Antibody-based therapy allows for avoidance or dose reduction of calcineurin inhibitors, possibly reducing the risk of nephrotoxicity. Regimens for immunosuppressive therapy are known in the art and are described in, for example, U.S. Pat. No. 9,561,253, the contents of which are incorporated herein by reference.

Immunosuppression may also diminish the response of the donor immune cells against recipient tissue, which can lead to GVHD. GVHD may be acute or chronic. Acute GVHD typically occurs in the first 3 months after graft and may involve the skin, intestine, or the liver. Treatment for acute GVHD usually includes high-dose corticosteroids such as prednisone. Chronic GVHD typically occurs after the first 3 months following transplant and is the major source of late treatment-related complications. Chronic GVHD may cause functional disability and require prolonged immunosuppressive therapy.

Immunosuppressive therapy may occur in multiple phases. For example, the immunosuppressive regimen may have an induction phase and a maintenance phase. Induction and maintenance phase strategies may use different medicines at doses adjusted to achieve target therapeutic levels to enhance engraftment of the CD34+ cells in the recipient.

Immunosuppressive therapy may be withdrawn after engraftment of the CD34+ cells has been established in the recipient. Advantageously, the regimens and compositions of the present invention greatly reduce the need to immunosuppressive therapies following organ donation and allow for immunosuppressive therapies to be withdrawn quickly after organ transplantation due to the establishment of engrafted CD34+ cells in the in the recipient. The CD34+ cell engraftment status of the recipient may be monitored as described below and deemed stable after a certain period, for example, 3 months, 6 months 12 months, 18 months, 24 months, or longer. Thus, immunosuppression may be discontinued for the recipients after a certain period, for example, 3 months, 6 months 12 months, 18 months, 24 months, or longer. Withdrawal of immunosuppressive therapy may include tapering, i.e., progressively reducing the dosage or frequency of treatment.

The present invention may also be utilized to engraft CD34+ cells in a manner that produces mixed chimerism in the recipient. A determination of whether an individual is a full chimera, mixed chimera, or non-chimera made be made by an analysis of a hematopoietic cell sample from the organ transplant recipient, e.g., peripheral blood, bone marrow, etc. as known in the art. Analysis may be done by any convenient method of typing. Analysis may be performed on hematopoietic cells or a subset thereof, such as all mononuclear cells, T cells, B cells, CD56⁺NK cells, and CD15⁺ neutrophils. Chimerism can be assessed by PCR analysis of microsatellites. For example, commercial kits that distinguish polymorphisms in short terminal repeat lengths of donor and host origin are available. Automated readers provide the percentage of donor type cells based on standard curves from artificial donor and host cell mixtures.

Recipients may be categorized as fully chimeric, mixed chimeric, or non-chimeric based on the fraction of cells that are derived from the donor. For example, recipients can be deemed fully chimeric if they have at least 90%, at least 95%, at least 98%, or at least 99% donor-derived cells. Recipients can be deemed mixed chimeric if they have too few donor-derived cells to be categorized as fully chimeric but a fraction of donor-derived cells that exceeds a certain threshold, such as at least 0.5%, at least 1%, at least 2%, at least 3%, at least 5%, at least 7.5%, at least 10% donor-derived cells. Recipients can be deemed non-chimeric if the fraction of donor-derived cells falls below the threshold required to be categorized as mixed chimeric.

### EXAMPLES

Preclinical studies showed that conditioning with TLI and ATG is advantageous for inducing tolerance after combined organ and bone marrow transplantation or HLA matched cell transplantation because the conditioning regimen prevents GVHD as compared to TBI. Approximately 1,000-fold more donor T cells are needed to induce lethal GVHD using TLI/ATG as compared to TBI conditioning. The basis of protection against GVHD is the change in the balance of residual host T cells that favors the host natural killer (NK) T cell subset. The latter cells become the predominant T cell subset in TLI/ATG conditioned mice and produce large amounts of IL-4 that polarize conventional donor T cells toward a Th2 bias, thereby attenuating GVHD. Several laboratories have shown that NK T cells that survive in vivo irradiation are themselves polarized toward a Th2 bias.

Based on the protective effect of TLI/ATG against GVHD in the preclinical studies, the presently disclosed methods incorporating a plurality of TBI doses is tested.

Twenty patients are given combined HLA matched or mismatched grafts and followed up from between 5 to 86 months to assess whether they develop mixed chimerism, including persistent chimerism, without the development of GVHD.

A gene array testing indicates that there is a change in the pre- to post-transplant gene expression profiles in patients who met drug withdrawal criteria such that their profiles became considerably better matched to those of the rare “operationally” tolerant patients who stop conventional immunosuppressive drugs and do not develop graft rejection at their first monitoring time point.

The study is performed in adult renal transplant patients. Patients receive a plurality of both TLI and TBI doses, RATG and an infusion of G-CSF “mobilized blood” mononuclear cells that had been enriched for CD34+ cells and contained a defined number of CD34+ and CD3+ donor cells. Immunosuppressive drugs consist of 9 months of mycophenolate mofetil (MMF; 15 mg/Kg twice per day starting on day 10), and a tapering course of daily tacrolimus starting on day 0 that is discontinued at a target of 12 months. The immunosuppressive drug combination of a calcineurin inhibitor and a purine metabolism inhibitor is similar to that used previously. At serial time points (1) graft function is monitored, (2) chimerism is measured in recipient white blood cell subsets and (3) protocol biopsies of the graft is obtained. If chimerism fails to develop or is lost during the first six months, or if a rejection episode or GVHD occurs or if there was histological evidence of rejection in graft biopsies then tacrolimus and/or MMF is not withdrawn, and the patient will be followed thereafter as a treatment failure. Recipients in the study are given a target dose of >10×10⁶ CD34+ cells/Kg and an escalating dose of T cells to achieve the target dose of 1×10⁶ cells/Kg T cells from the “mobilized” peripheral blood mononuclear cells harvested from the donor.

During the course of this study, patients receive 5 intravenous injections of rabbit ATG (Thymoglobulin), multiple doses of TLI and TBI, a single infusion of donor cells, transient immunosuppression (MMF and Tacrolimus), and prophylactic anti-viral, anti-fungal and antibacterial agents.

Patients receive a total of 5 intravenous doses of Thymoglobulin over a 5-day period; each dose is 1.5 mg/kg. Thymoglobulin is administered on the day of transplantation (intra-operatively before the transplanted organ is perfused with host blood) and on the subsequent 4 days post-transplant.

Patients receive between 1 and 10 doses of TLI targeted to the lymph nodes, spleen and thymus gland on days 1 through 4, and days 7 through 11, and at least two doses of TBI during those 11 days. Two doses may be given on a single. TLI is given to the inverted Y and mantle fields. During the administration of TLI, patients are monitored for the development of neutropenia (granulocytes<2,000/mL), thrombocytopenia (platelets<60,000/mL) and infection. TLI is withheld for any of these problems, and G-CSF (10 μg/kg/day) is given for neutropenia. TLI is reinstated once neutropenia and/or thrombocytopenia resolves. At the completion of TLI, all patients are given G-CSF (10 μg/kg/day) if the white blood cell count is below 1,000 cells/mm3. TL is completed by day 11 if no doses are withheld.

A single intravenous infusion of cryopreserved HLA-haplotype matched living related donor, G-CSF mobilized blood mononuclear cells (recovered from donor peripheral blood using apheresis), that are “engineered” is administered to patients on the day of completion of the final dose of irradiation, which is generally a dose of TBI. Harvesting of donor cells is performed in the following fashion. Approximately 6 weeks before renal transplantation, the donor is given G-CSF daily (16 mg/kg/day) for five days, and mononuclear cells are harvested by an apheresis of up to 20 liters according to procedures previously approved by the Stanford Committee on Medical Human Subjects for HLA-haplotype matched peripheral blood stem cell (PBSC) transplantation. In addition, a second session of up to 12 liters may be carried out for optimal cell recovery. Cells are selected for CD34+ cells on Isolex columns, Column flow through is collected also. Both CD34+ cells and flow through cells are cryopreserved and thawed according to standard procedures at the Stanford Blood and Marrow Transplantation laboratory. The target dose of CD34+ cells to be injected is >10×10⁶ cells/kg. A defined target dose of (1×107/kg CD3+) T cells is administered by injecting column flow through cells along with enriched CD34+ cells intravenously. The dose of flow through cells are calculated based on the content of CD3+ cells determined by immunofluorescent staining. This dose was used in the 17 haplotype matched patients with leukemia and lymphoma. The dose of T cells is increased if the first 3 recipients of combined transplants fail to achieve persistent chimerism, and is decreased if the first 3 recipients develop complete chimerism or if any patient develops GVHD.

Corticosteroid therapy is limited to 60-120 mg Solumedrol (I.V.) as premedication on the days of ATG infusions to reduce ATG side effects. After the last dose of ATG, a tapering course of prednisone starting at 30 mg/d and reducing by 5 mg/d is given until day 10.

MMF therapy will commence on the day of the donor cell infusion (day 10) at 15 mg/Kg twice per day MMF therapy is maintained for 6 months, and then tapered and stopped at 9 months.

Tacrolimus is started on day 0, adjusted to achieve a standing whole blood trough level. As long as the criteria for immunosuppressive drug tapering are met, Tacrolimus is tapered beginning at month 9, and stopped by month 12.

Criteria for continued tapering of immunosuppressive drugs (Tacrolimus) through month 12 and MMF through month 9 is as follows: 1) Sustained chimerism for at least 6 months, 2) No clinical rejection episodes; 3) Protocol biopsies show no evidence of acute or chronic rejection; 4) No GVHD. Patients who do not meet these criteria are considered treatment failures, and further tapering of drugs is not performed.

If acute or chronic GVHD is observed that would ordinarily be treated with immunosuppressive drugs, then the patient is considered a treatment failure. Immunosuppressive drugs are administered according to standard practice.

Rejection episodes are treated with standard anti-rejection therapy which includes the use of intravenous methyl prednisolone and the patient is considered a treatment failure. If no response to two courses of steroids is found, then a course of anti-lymphocyte antibody is given. Tacrolimus is given at conventional doses during rejection episodes. Once a rejection episode occurs, patients will return to conventional doses of maintenance immunosuppressive drugs and no fuirther tapering is attempted as per the protocol above. Currently 95% of acute rejection episodes are reversed.

Since the initial course of TLI and ATG is expected to induce a marked depletion of T cells, there is an increased risk of new or recrudescent viral infection, including cytomegalovirus (CMV), Epstein-Barr virus, Herpes zoster and Herpes simplex viruses as compared to conventional immunosuppressive protocols. Antiviral prophylaxis for CMV is given as follows. Valganciclovir (900 mg/d:P.O.) is given for the first 14 days adjusted for renal function or during the first 14 days, ganciclovir (DHPG), 5 mg/kg, is given IV adjusted for renal function. After the 14-day course all protocol transplant recipients are placed on the valganciclovir (900 mg/d) adjusted for renal function. This is continued for a minimum of 90 days and if the absolute lymphocyte count is under 400, is continued until the time of steroid discontinuation.

Bactrim (1 single strength tablet per day) was given orally for one year for prophylaxis of Pneumocystis carinii pneumonia (PCP) and Mycostatin mouthwashes daily for three weeks for Candida prophylaxis. Standard peri-operative antibiotics will include Ancef (1 mg, I.V., 3 doses at 8-hour intervals) and Gentamicin (1.7 mg/kg, I.V., one dose at the time of transplant). Antibacterial agents are subject to appropriate substitution according to patient allergies.

A surveillance biopsy is performed just before all immunosuppressive drugs are stopped posttransplant. In addition, “for-cause” biopsies are obtained within 48 hours of an unexplained or unresolved 20% increase in serum creatinine.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method for transplantation, the method comprising: conditioning a recipient of an organ transplant with a plurality of total lymphoid irradiation (TLI) doses, and at least two doses of total body irradiation (TBI), each dose of TBI between about 15 and about 75 cGy; infusing the recipient with a donor-derived hematopoietic stem cell product; wherein the recipient achieves stable, high level mixed-chimerism with the donor hematopoietic cells.

2. The method of claim 1, wherein two doses of TBI are provided to the recipient.

3. The method of claim 1, wherein three or more doses of TBI are provided to the recipient.

4. The method of claim 1, wherein the plurality of TLI doses consists of between 1 and 11 doses.

5. The method of claim 1, wherein the plurality of TLI doses comprise a total dose of between about 1100 cGy and about 1300 cGy.

6. The method of claim 5, wherein each individual TLI dose comprises a dose of between about 130 cGy and about 150 cGy.

7. The method of claim 1, wherein the donor and recipient are MHC-mismatched.

8. The method of claim 1, the method further comprising administering one or more doses of anti-thymocyte globulin (ATG) to the recipient.

9. The method of claim 1, wherein a final dose of irradiation is one or more of the TBI doses.

10. The method of claim 1, wherein the recipient receives a solid organ transplantation.

11. The method of claim 10, wherein the donor donates the solid organ.

12. The method of claim 1, wherein the hematopoietic cellular product comprises CD34+ cells.

13. The method of claim 12, wherein the hematopoietic cellular product further comprises CD3+ cells.

14. The method of claim 13, wherein the hematopoietic cellular product comprises at least 1×10⁵ CD34+ cells/kg recipient weight and at least 1×10⁵ CD3+ cells/kg recipient weight.

15. The method of claim 14, wherein the hematopoietic cellular product comprises at least 1×10⁶ CD34+ cells/kg recipient weight and at least 1×10⁶ CD3+ cells/kg recipient weight.

16. The method of claim 15, wherein the hematopoietic cellular product comprises at least 4×10⁶ CD34+ cells/kg recipient weight and at least 1×10⁸ CD3+ cells/kg recipient weight.

17. The method of claim 12, wherein the hematopoietic cellular product further comprises CD8+ memory T cells.

18. The method of claim 17, wherein the hematopoietic cellular product comprises at least 1×10⁶ CD8+ memory T cells/kg recipient weight.

19. (canceled)

20. The method of claim 1, wherein the donor is deceased.

21. The method of claim 1, wherein prior to the infusion of the hematopoietic stem cell product, the recipient receives a solid tissue or organ transplant.

22-23. (canceled)
